(19) United States
(12) Patent Application Publication
(10) Pub. No.: US 2017/0022540 A1
(43) Pub. Date: Jan. 26, 2017

(54) BIOINK FOR THREE-DIMENSIONAL BIOMATERIAL PRINTING

(71) Applicant: David Fries, St. Petersburg, FL (US)

(72) Inventor: David Fries, St. Petersburg, FL (US)

(73) Assignee: University of South Florida, Tampa, FL (US)

(21) Appl. No.: 15/218,199

(22) Filed: Jul. 25, 2016

Related U.S. Application Data
(60) Provisional application No. 62/196,727, filed on Jul. 24, 2015.

(51) Int. Cl. C12Q 1/68; C12N 9/96; C09D 201/00; C09D 11/02; C09D 189/00; C12N 5/00; C09D 11/04

(52) U.S. Cl. CPC C12Q 1/686; C12N 5/00; C12N 9/96; C09D 11/04; C09D 11/02; C09D 189/00; C09D 201/00

(57) ABSTRACT

A method of creating “bioink” has been proposed, which increases the shelf-life and transportability of biological materials for use in bioprinting applications. The current invention also contemplates a composition of biomaterials admixed with trehalose to dehydrate the biomaterials for preservation and transportation. The dehydrated biomaterials can then be rehydrated for use in bioprinting.

### CROSS-REFERENCE TO RELATED APPLICATIONS

This nonprovisional application claims priority to U.S. Provisional Patent Application No. 62/196,727, entitled “Bioink for Three-Dimensional Biomaterial Printing”, filed Jul. 24, 2015 by the same inventor, the entirety of which is incorporated herein by reference.

### BACKGROUND OF THE INVENTION

1. Field of the Invention

This invention relates, generally, to bioprinting. More specifically, it relates to bioinks for biosystems printing.

2. Brief Description of the Prior Art

Bioprinting is the application of three-dimensional inkjet printing techniques for the patterning and deposition of biological materials. Cell patterns are created layer-by-layer, such that cell function and viability are preserved in the resulting printed construct and can be used in medical and tissue engineering. Currently, there is interest in the ability to pattern tissues, organs, proteins, biomolecules, cells, and tissue scaffolds, among other biomaterials. However, one of the major challenges with bioprinting is the ability to preserve the biological materials to be printed. When the biological materials break down, they have shown decreased bioavailability and functioning, particularly in critical components that are needed for the body's normal biochemical processes.

Accordingly, what is needed is an improved methodology for preserving and transporting biological materials for bioprinting. However, in view of the art considered as a whole at the time the present invention was made, it was not obvious to those of ordinary skill in the field of this invention how the shortcomings of the prior art could be overcome.

While certain aspects of conventional technologies have been discussed to facilitate disclosure of the invention, Applicants in no way disclaim these technical aspects, and it is contemplated that the claimed invention may encompass one or more of the conventional technical aspects discussed herein.

The present invention may address one or more of the problems and deficiencies of the prior art discussed above. However, it is contemplated that the invention may prove useful in addressing other problems and deficiencies in a number of technical areas. Therefore, the claimed invention should not necessarily be construed as limited to addressing any of the particular problems or deficiencies discussed herein.

In this specification, where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned.

### BRIEF SUMMARY OF THE INVENTION

The long-standing but heretofore unfulfilled need for an improved method of preserving biomaterials is now met by a new, useful, and nonobvious invention.

In an embodiment, the current invention is a dehydrated bioink composition, comprising biomaterials admixed with a clinically effective amount and concentration of trehalose.

In other embodiments, the current invention is a method of preserving and using biomaterials for bioprinting. Biomaterials are dehydrated by admixing them with a clinically effective amount and concentration of trehalose, where the dehydrated biomaterials are suitable for preservation and transportation. To bioprint then, the dehydrated biomaterials can be rehydrated, and the rehydrated biomaterials would be printed.

Between dehydrating and rehydrating the biomaterials, the mixture of the biomaterials and trehalose may be loaded onto a cryogenic plate via dripping, spraying, or contact transfer. The cryogenic plate can be achieved by a cryogenic pump, pumped liquid nitrogen, or a cold finger. The cryogenic is motioned to yield a mass load and a pattern, and the cryogenic plate is configured as hydrophobic or hydrophilic to allow for the release of a printed ink from the mixture of the biomaterials and trehalose.

These and other important objects, advantages, and features of the invention will become clear as this disclosure proceeds.

The invention accordingly comprises the features of construction, combination of elements, and arrangement of parts that will be exemplified in the disclosure set forth hereinafter and the scope of the invention will be indicated in the claims.

### BRIEF DESCRIPTION OF THE DRAWINGS

For a fuller understanding of the invention, reference should be made to the following detailed description, taken in connection with the accompanying drawings, in which:

FIG. 1 is a graphical illustration depicting an example of efficacy of trehalose stabilized proteins/enzymes and nucleic acids.

FIG. 2 depicts an exemplary device for carrying out a methodology according to an embodiment of the current invention.

FIG. 3 depicts an exemplary system for making bioinks using a cold plate system, according to an embodiment of the current invention.

### DETAILED DESCRIPTION OF THE PREFERRED EMBODIMENT

In the following detailed description of the preferred embodiments, reference is made to the accompanying drawings, which form a part thereof, and within which are shown by way of illustration specific embodiments by which the invention may be practiced. It is to be understood that other embodiments may be utilized and structural changes may be made without departing from the scope of the invention.

As used in this specification and the appended claims, the singular forms “a”, “an”, and “the” include plural referents unless the content clearly dictates otherwise. As used in this specification and the appended claims, the term “or” is generally employed in its sense including “and/or” unless the context clearly dictates otherwise.

Trehalose, also known as mycose or tremalose, is a sugar implicated in anhydrobiosis, or desiccation tolerance. Trehalose has a high water retention rate, so when it is introduced to another material, it can draw material out of that other material. Trehalose is thought to form a gel when cells dehydrate which prevents the disruption of internal cell organelles. When cells are allowed to rehydrate, normal cellular activity resumes without major damage.

Trehalose has been used in various ways, such as those seen in U.S. Pat. No. 8,921,085; Chinese Patent No. CN103768657; U.S. Patent Application Publication No. 2005/0048648; Nishioka, Gary M., et al. “Protein damage in drop-on-demand printers.” Journal of the American Chemical Society 126.50 (2004): 16320-16321; Yamada, Misato, et al. “Microarrays of phospholipid bilayers generated by inkjet printing”, Langmuir 29.21 (2013): 6404-6408; and Brandstetter, T. “Biochip Technologies Biochip-Technologies”. However, the foregoing references suffer from one or more of the drawbacks previously noted, in that none teach or result in the sufficient preservation needed for bioprinting.

As discussed herein and will become clearer as this specification continues, biological materials—when admixed with a sufficient amount and concentration of trehalose—can be dehydrated and preserved for future use in bioprinting, thereby creating a “bioink”. The bioink would have an improved shelf-life for biological materials and simplify its transport. When ready for deposition, the bioink can be rehydrated and printed.

Study

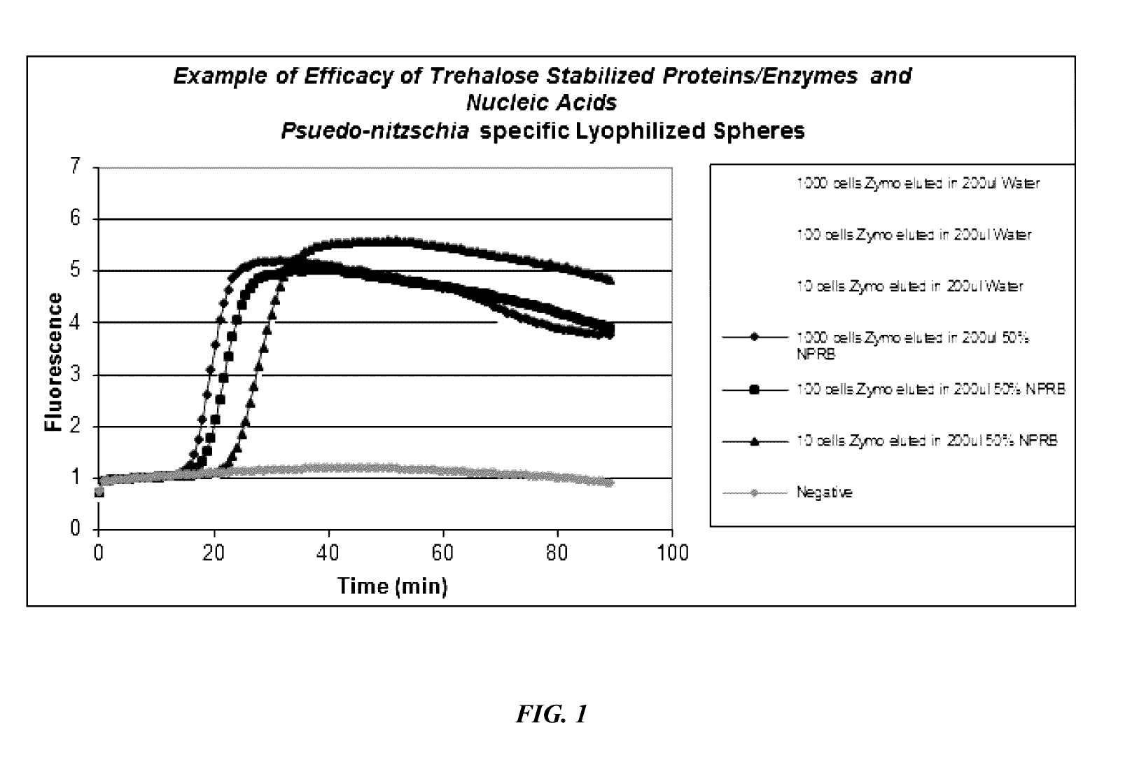

In an embodiment, the current invention is bioink lyophilized NASBA (nucleic acid sequence based amplification) RNA amplification mix (contains AMV Reverse Transcriptase, T-7 RNA Polymerase RNase H, and Genetic Primers). The following presents validation and processing platform implementation. An example assay, Pseudo nitzschia, was used for validation. The combined protein and genetic molecule based assay was refined and its specificity was determined. Conducting a manual extraction test, the results depicted FIG. 1 were obtained.

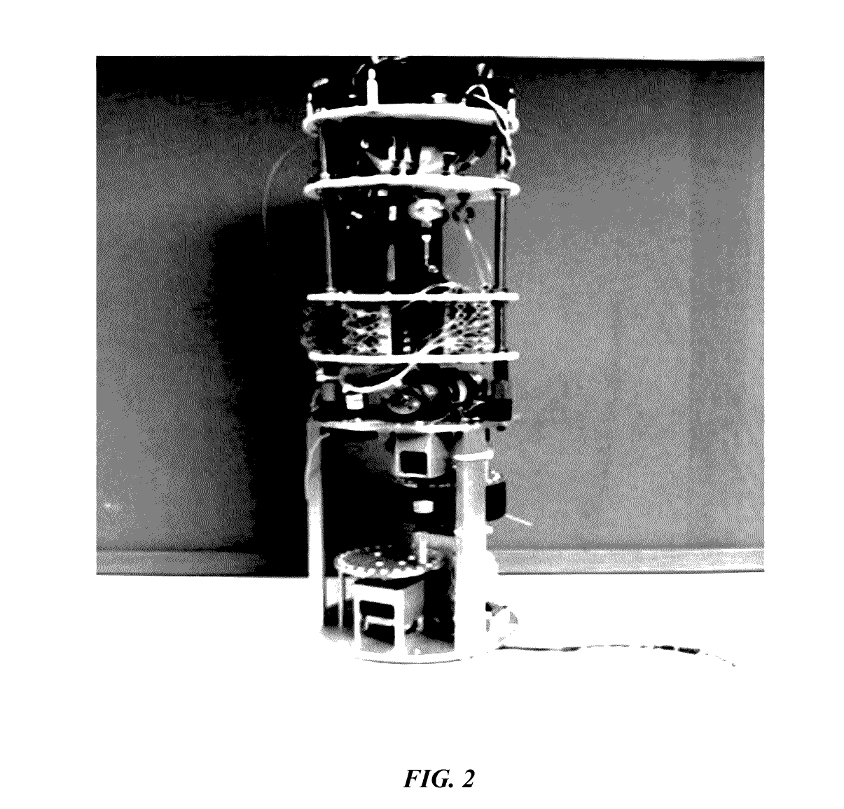

In the approach (see the device of FIG. 2), primers were tested with target and non-target species. Single reaction spheres (lyophilized bioinks) were then developed and tested. The inks can be made either by dripping droplets of reagents into liquid N₂ or by variable loading printing onto a cryo-cold plate. As an additional trehalose sphere test format, automated fluidic processors can be used on the bioink sphere to reconstitute and validate efficacy.

### Example

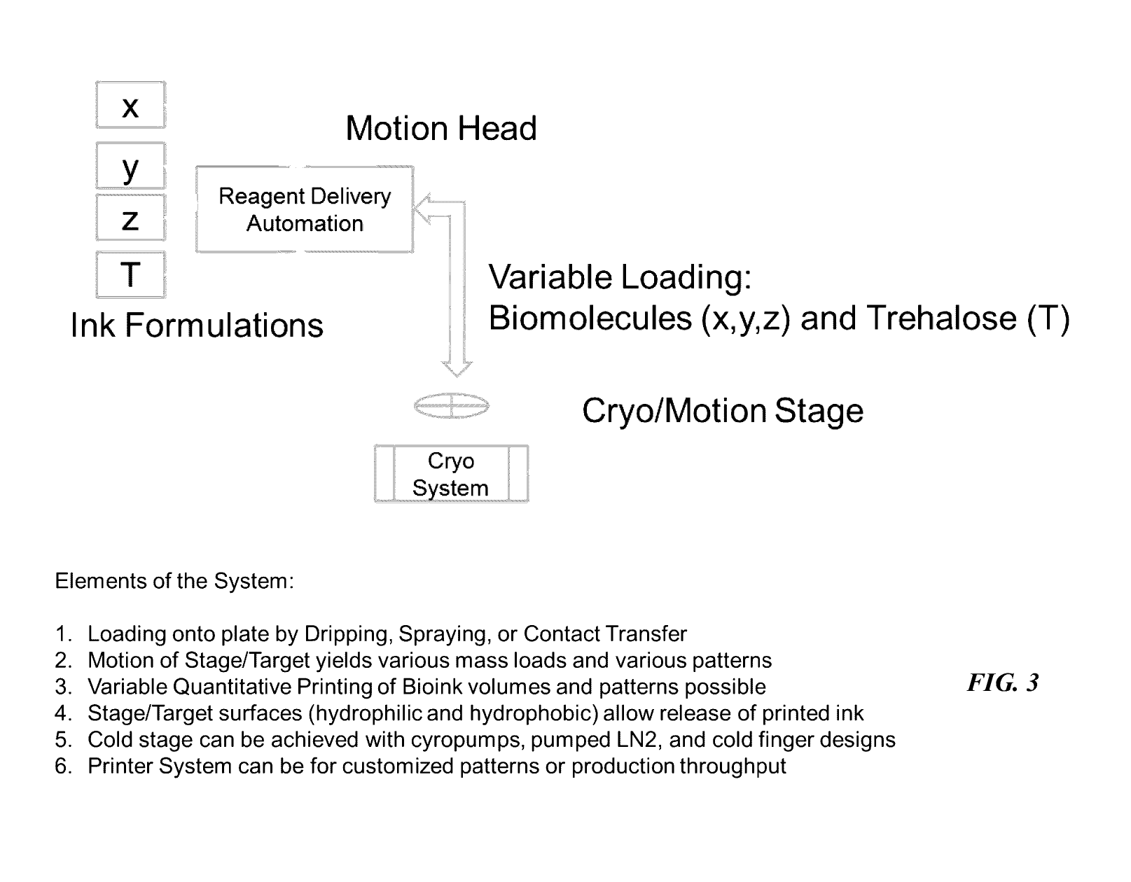

Presented herein is an example of an apparatus that can be used to make such bioinks using a cold plate system. The elements of the system can be seen in FIG. 3. The plate is loaded by dripping, spraying, contact transfer, or other methodology known in the art. Motion of the stage/target yields various mass loads and various patterns. Varying the quantitative printing of bioink permits different volumes and patterns possible. A further element is that the stage/target surfaces can be configured as (hydrophilic and hydrophobic) and allow for the release of the printed ink. The cold stage can be achieved with cryopumps, pumped liquid N₂, and/or cold finger designs. The printer system as a whole can be used for customized patterns and/or production throughput.

Glossary of Claim Terms

Bioink composition: This term is used herein to refer to a substance made from living cells for printing tissues, organs, proteins, biomolecules, cells, and tissue scaffolds, among other suitable bioprinted constructs.

Biomaterials: This term is used herein to refer to a synthetic or natural material for use in constructing different organs or tissues.

Bioprinting: This term is used herein to refer to the creation of patterns of living cells, where cell function and viability are preserved within the resulting construct (e.g., organ, tissue, etc.).

Clinically effective: This term is used herein to refer to concentrations or amounts of components such as agents (e.g., trehalose) which are effective for producing an intended result, including dehydration of a biomaterial. Compositions according to the present invention may be used to effect a favorable change in the hydration levels of biomaterials. Optimization of such factors (amount and concentration) is well within the level of skill in the art, unless otherwise noted.

Cryogenic plate: This term is used herein to refer to a stage maintained at very low temperatures.

The advantages set forth above, and those made apparent from the foregoing description, are efficiently attained. Since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention that, as a matter of language, might be said to fall therebetween.

What is claimed is:

1. A bioink composition, comprising biomaterials admixed with a clinically effective amount and concentration of trehalose.

2. A method of preserving and using biomaterials for bioprinting, comprising:
dehydrating biomaterials by admixing said biomaterials with a clinically effective amount and concentration of trehalose, said dehydrated biomaterials being suitable for preservation and transportation.

3. A method as in claim 2, further comprising rehydrating said dehydrated biomaterials and printing said rehydrated biomaterials.

4. A method as in claim 2, further comprising:
loading the admixture of said biomaterials and said trehalose onto a cryogenic plate via dripping, spraying, or contact transfer,
said cryogenic plate achieved by a cryogenic pump, pumped liquid nitrogen, or a cold finger;
motioning said cryogenic plate to yield a mass load and a pattern; and
configuring said cryogenic plate as hydrophobic or hydrophilic to allow for a release of a printed ink from said admixture of said biomaterials and said trehalose.
